(19) United States
(12) Patent Application Publication
(10) Pub. No.: US 2020/0289761 A1
(43) Pub. Date: Sep. 17, 2020

(54) SYRINGE HAVING AT LEAST ONE RADIALLY-OUTWARDLY EXTENDING PANEL

(71) Applicants: Merck Sharp & Dohme Pharmaceutical Industrial and Commercial Societe Anonyme, Athens (GR); MSD IT Global Innovation Center S.R.O., Prague 5 (CZ)

(72) Inventors: Georgios Errikos GRIGOREAS, Pefki (GR); Ashley Marie POLHEMUS, Prague (CZ)

(73) Assignees: Merck Sharp & Dohme Pharmaceutical Industrial and Commercial Societe Anonyme, Athens (GR); MSD IT Global Innovation Center S.R.O., Prague 5 (CZ)

(21) Appl. No.: 16/767,757

(22) PCT Filed: Nov. 26, 2018
(86) PCT No.: PCT/US2018/062416

§ 371 Date: May 28, 2020

Related U.S. Application Data
(60) Provisional application No. 62/660,479, filed on Apr. 20, 2018.

(30) Foreign Application Priority Data
Dec. 1, 2017 (GR) 20170100548

(51) Int. Cl. A61M 5/315; A61M 5/31

(52) U.S. Cl. CPC A61M 5/3156; A61M 2005/3126; A61M 5/3129

(57) ABSTRACT

A syringe is provided herein for injecting medication into a patient, the syringe including a tubular barrel having opposing inner and outer surfaces, an open proximal end, and a distal end having a wall extending thereacross. An opening is defined in the wall with a needle mount extending distally from the wall about the opening. A first panel extends radially outwardly from the outer surface of the barrel, the first panel having opposing first and second faces. Further, a first scale including a series of spaced-apart first graduations is disposed on the first face of the first panel. Advantageously, with the subject invention, a syringe may be provided having a planar portion, in addition to its barrel, with graduations marked thereon for improved viewing by a practitioner in determining a proper dose amount.

### FIELD OF THE INVENTION

The subject invention relates to syringes.

### BACKGROUND OF THE INVENTION

Syringes are well known in the art for administering injections. With plastic disposable syringes, there is a need to aspirate an amount of medication from a vial or other drug container with subsequent injection into a patient. This process may require calculations or other determinations (e.g., table or chart look-ups) for a medical practitioner to determine a proper dose, as the dose may be based on a physiological parameter of a patient, such as the patient's weight. Such injections, as opposed to fixed-dose amounts, run the risk of possible error.

Arrangements have been provided in the prior art to simplify the calculation or determination of an unknown dose, wherein scales have been provided with a syringe in units of a target physiological characteristic, such as a patient's weight. This allows for a practitioner to administer medication based on the provided scale, rather than requiring the calculation or determination of a dose in fixed units of volume, such as milliliters, cubic centimeters, etc. For example, U.S. Pat. No. 9,566,388 to Jones provides a plunger for a syringe having disposed thereon a scale in units of a patient's weight. WO 2016/176523 discloses a transparent sleeve that is mountable to a standard syringe with the sleeve having thereon scales related to weight and other patient characteristics.

### SUMMARY OF THE INVENTION

A syringe is provided herein for injecting medication into a patient, the syringe including a tubular barrel having opposing inner and outer surfaces, an open proximal end, and a distal end having a wall extending thereacross. An opening is defined in the wall with a needle mount extending distally from the wall about the opening. A first panel extends radially outwardly from the outer surface of the barrel, the first panel having opposing first and second faces. Further, a first scale including a series of spaced-apart first graduations is disposed on the first face of the first panel. Advantageously, with the subject invention, a syringe may be provided having a planar portion, in addition to its barrel, with graduations marked thereon for improved viewing by a practitioner in determining a proper dose amount.

As used herein, the term “proximal”, and derivatives thereof, refers to a direction away from a patient. In addition, as used herein, the term “distal”, and derivatives thereof, refers to a direction towards a patient.

These and other features of the invention will be better understood through a study of the following detailed description and accompanying drawings.

### BRIEF DESCRIPTION OF THE DRAWINGS

FIGS. 1-7 depict an embodiment of a syringe with a first panel formed in accordance with the subject invention; and,

FIGS. 8-13 depict an embodiment of a syringe with first and second panels formed in accordance with the subject invention.

### DETAILED DESCRIPTION OF THE INVENTION

With reference to the Figures, a syringe 10 is shown and generally designated with the reference numeral 10. The syringe 10 generally includes a tubular barrel 12 and a first panel 14, with a first scale 16 being disposed on the first panel 14. With this embodiment, and with the various additional embodiments described herein, the scale may be provided on a panel having a planar surface or a curved surface. In preferred embodiments, the scale is provided on a panel having a planar surface for improved viewing by a practitioner. The syringe 10 is for injection of a medication into a patient. As used herein, a “medication” refers to any injectable liquid having one or more active pharmaceutical and/or biological ingredients.

The syringe 10 may be configured and dimensioned as any syringe. The syringe 10 is particularly well-suited to serve as a plastic, disposable syringe, with the barrel 12 being formed of thermoplastic. The barrel 12 and the first panel 14 may be unitarily formed using any technique such as injection molding. The syringe 10 may be used to aspirate a medication from a vial or other drug container in an amount as needed, with the syringe 10 being subsequently used to administer an injection of medication. The barrel 12 acts as a reservoir for the medication.

The barrel 12 includes opposing inner and outer surfaces 18, 20. Proximal end 22 of the barrel 12 is open while distal end 24 of the barrel 12 includes wall 26 extending thereacross. An opening 28 is provided in the wall 28 through which medication may be delivered. Needle mount 30 is provided extending distally from the wall 26 about the opening 28. The needle mount 30 may be any needle mount useable for securement of a needle for injection, including a luer tip 30A mount, optionally, in combination with threaded mount 30B, such as in a luer lock configuration. The needle mount 30 may be used with any needle assembly 32 having a needle 34, with a lumen 36 and a sharpened distal tip 38 for insertion into a patient. The needle assembly 32 includes a hub 40 for cooperatively mounting onto the needle mount 30 such that a liquid passageway 42 is defined from the opening 28 and through the lumen 36 to the distal tip 38 with the needle assembly 32 mounted onto the needle mount 30. The hub 40 may include a luer tip mount and threads, as necessary, to cooperatively engage the needle mount 30.

As known in the art, the inner surface 18 of the barrel 12 may be coated with a lubricant or other substance, e.g., for compatibility with the medication. In addition, the wall 26 may be provided as funnel-shaped configured to direct flow towards the opening 28.

A flange 44 may be provided on the outer surface 20 of the barrel 12 about the proximal end 22. The flange 44 may include first and second flange portions 46, 48 which protrude in opposite directions. As appreciated by those skilled in the art, the first and second flange portions 46, 48 may be shaped and sized to be pressingly engaged by a user's index and middle fingers, respectively, during an injection. A plunger 50 may be provided sized to pass through the proximal end 22 and into the barrel 12. The plunger 50 includes a plunger piston 52 at a distal end 54 thereof. The plunger piston 52 is preferably elastomeric and configured to sealingly engage the inner surface 18 of the barrel 12 with sliding movement relative thereto for aspirating into, and urging from, the barrel 12 medication via the opening 28 as a result of proximal or distal advancement of the plunger piston 52 within the barrel 12. A thumb pad 57 may be provided at a proximal end 59 of the plunger 50.

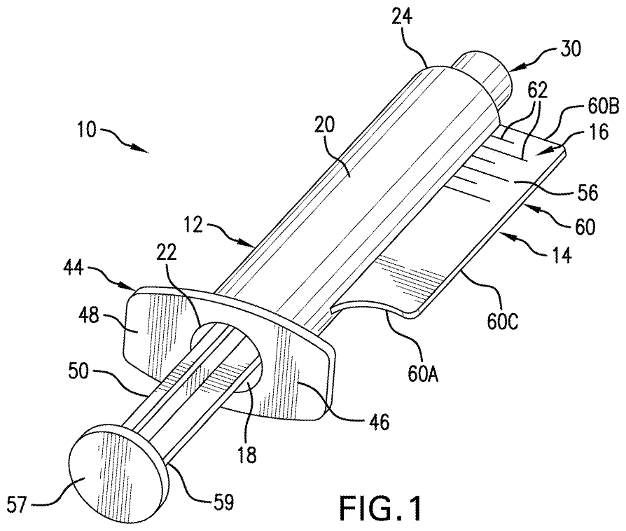

With reference to FIGS. 1-5, the first panel 14 may be provided to extend radially outwardly from the outer surface 20 of the barrel 12. The first panel 14 includes opposing first and second faces 56, 58. Preferably, the first panel 14 is generally plate-shaped with the first and second faces 56, 58 being generally parallel.

The first panel 14 may include a plurality of edges 60 extending between the first and second faces 56, 58. A first edge 60A may face proximally, with a second edge 60B facing distally. A third edge 60C may extend between the first and second edges 60A, 60B. The first edge 60A is preferably spaced distally from the flange 44 to define a space therebetween. Preferably, the space is sufficient to accommodate a user's index or middle finger during use.

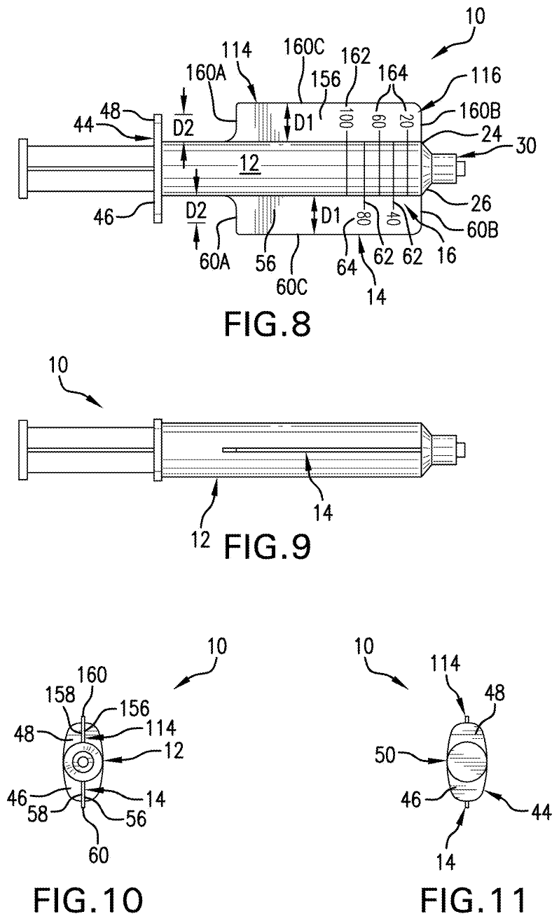

It is preferred that the syringe 10 overall be provided with a profile which allows for disposal in a standard sharps container (i.e., biohazard container for used syringes). As such, it is preferred that the first panel 14 be limited in radial extent from the barrel 12. This radial extent may be characterized relative to the size of the flange 44. In particular, as shown in FIG. 8, the third edge 60C may be located maximally at a first distance D1 from the outer surface 20 of the barrel 12 with the first flange portion 46 extending maximally a second distance D2 from the outer surface of the barrel 12. The first distance D1 is preferably in the range of 80%-150% of the second distance D2. It is also preferred that the first panel 14 be radially aligned with the first flange portion 46.

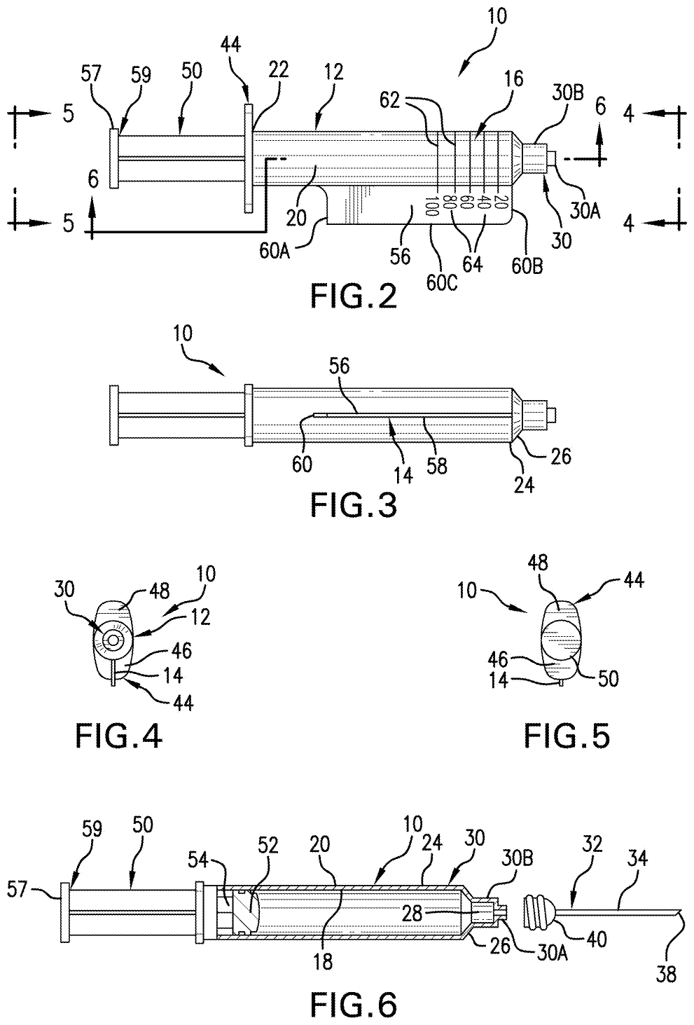

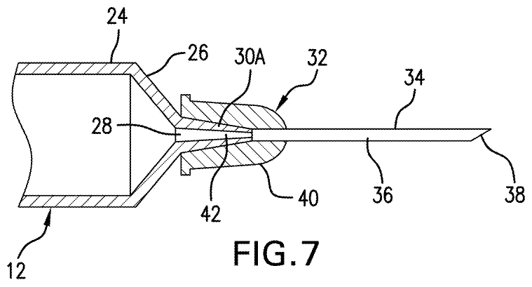

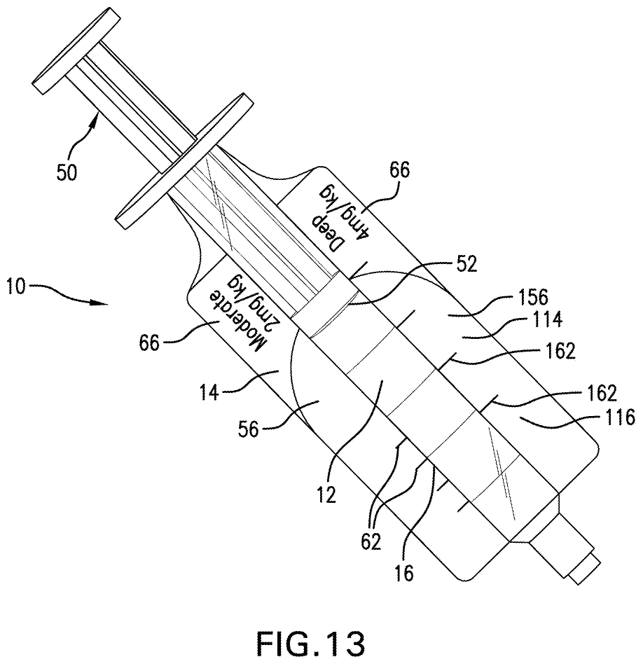

The first scale 16 is disposed on the first panel 14, particularly, on the first face 56 thereof, as shown in FIG. 1. The first scale 16 includes a series of first graduations 62, spaced at intervals which may be regular or irregular. The first graduations 62 are generally perpendicular to the barrel 12. As shown in FIG. 2, the first graduations 62 preferably extend uninterruptedly onto the barrel 12 particularly to extend about a portion, possibly about the entirety, of the circumference thereof, e.g., along the outer surface 20. The barrel 12 is preferably transparent or sufficiently translucent to allow for a user to see the plunger piston 52 therewithin. As shown in FIG. 13, this arrangement allows for a user to visually align the piston plunger 52 with the first graduations 62 to aspirate a target amount into the barrel 12 for dosing and/or administer a target dose from the barrel 12. With the first graduations 62 being presented on at least the first panel 14, more precise alignment between the piston plunger 52 and a targeted first graduation 62 may be achieved.

Indicia 64 may be provided with the first scale 16 to assign numerical values to some or all of the first graduations 62. This allows for a user to identify amounts. The indicia 64 may be arranged to increase in value in a proximal direction so that the first scale 16 increases in magnitude in a proximal direction.

The first scale 16 may be presented based on one or more physiological parameters related to the medication and/or the patient intended for injection. The physiological parameters may be selected on the basis of one or more of: age of a patient, gender of a patient, actual weight of a patient, ideal weight of a patient, body surface area of a patient, indication to be treated, severity of a patient's condition, level of impairment of a patient's one or more organs, concentration of the medication being administered, patient's resistance (or tolerance) to a medication, pharmacokinetics of a medication, and allometrically scaled dosing of a medication. The first scale 16 may have the first graduations 62 spaced at intervals based on the physiological parameter(s) with the indicia 64 presenting associated values. For example, the first graduations 62 may be spaced apart for a particular medication at intervals corresponding to target amounts based on a patient's weight, with the indicia 64 presenting different possible weights (in any units, such as pounds, kilograms, and so forth).

As shown in FIGS. 8-13, a second panel 114 may be provided with the syringe 10 which includes the same characteristics as the first panel 14. The numbering from the first panel 14 has been applied to the second panel 114, but with the addition of 100 and the modifier “second” or “secondary”, so that reference number 14 (first panel) corresponds to reference number 114 (second panel) and so forth. Unless noted otherwise, all elements for the second panel 114 are the same as in the first panel 14, with the configuration, dimensioning and spacing of the elements in the first panel 14 applying equally to the elements of the second panel 114.

The second panel 114, as best shown in FIG. 10, is preferably aligned so that the first face 56 and the first secondary face 156 both face generally in the same direction. The second panel 114 may extend from the outer surface 20 of the barrel 12 in a diametrically opposing direction from the first panel 14. The second panel 114 may be distally spaced from the flange 44 at the same, or a different, distance as the first panel 14 is spaced from the flange 44. It is preferred that the second panel 114 be sufficiently spaced from the flange 44 to accommodate a user's index or middle finger during use. Also, the second panel 114 includes the secondary third edge 160C which may be located maximally at a first distance D1 from the outer surface 20 of the barrel 12 with the second flange portion 48 extending maximally a second distance D2 from the outer surface of the barrel 12. The first distance D1 is preferably in the range of 80%-150% of the second distance D2. It is also preferred that the first panel 14 be radially aligned with the first flange portion 46. The first distance D1 for the third edge 60C may be the same as or different from the first distance D1 for the secondary third edge 160C.

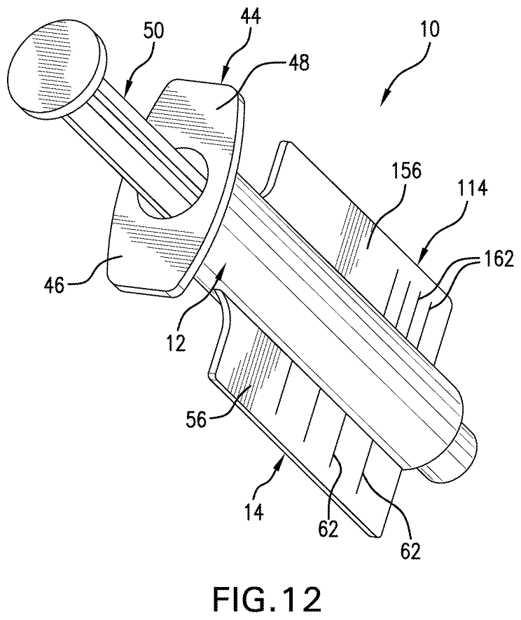

The second scale 116 may be disposed on the second panel 114, particularly, on the first secondary face 156 (FIG. 12). The second scale 116 may be based on one or more of the physiological parameters related to the medication and/or the patient intended for injection as discussed above in connection with the first scale 16. The second graduations 162 preferably extend interruptedly onto the barrel 12 particularly to extend about a portion of the circumference thereof, e.g., along the outer surface 20, as shown in FIG. 8. The first graduations 62 may extend to, and possibly onto, the second panel 114. In addition, the second graduations 162 may extend to, and possibly onto, the first panel 14.

The first and second scales, 16, 116 may be provided to be complementary, e.g., with the first and second graduations 62, 162 and the first and second indicia 64, 164 defining a combined, single scale, as shown in FIG. 8. Alternatively, the first and second scales 16, 116 may be provided as different scales with different intervals between the graduations to allow for multiple uses of the syringe 10, as shown in FIG. 13. For example, the first scale 16 may be established to represent dosing of a medication based on a patient's weight, whereas, the second scale 116 may be established to represent dosing of a medication based on a patient's body surface area. The same or different medications may be used.

The first and second graduations 62, 162 may be provided in part or in whole in different colors, and/or stylizations (shape, presentation) to be distinguishable on or near the barrel 12. Headers or other explanatory indicia 66 may be provided to indicate what each scale represents. In addition, either or both of the second face 58 and/or the second secondary face 158 may be utilized in combination with either or both of the first face 56 and/or the first secondary face 156. This allows for additional scales to be used, e.g., four different scales, one on each face of each panel. In addition, the first face 56 and the first secondary face 156 may have complementary scales, while the second face 58 and the second secondary face 158 also have complementary scales. This allows for a user to flip the syringe 10 to a particular orientation depending on the target dosing.

As will be appreciated by those skilled in the art, although the subject invention is discussed and shown with one or two panels, it is to be understood that additional panels may be used at various radial spacing about the barrel 12.

As indicated above, one or more scales (e.g., the first and/or second scales 16, 116) may be based on one or more physiological parameters related to the medication and/or the patient intended for injection. The following presents non-limiting examples of drugs and physiological parameters which may be used in determining the one or more scales. As also indicated above, where additional panels are used at various radial spacing about the barrel 12, additional scales may be shown on each face of each such panel. Each of the examples below can be configured according to any of the above described embodiments and configurations of syringe 10, as will be appreciated by those of ordinary skill in the art.

### Example 1: Differentiating Dose Markings by Indication

In some cases, a particular medication formulated for injection can be administered for two or more different indications, each at different dosing regimens depending upon the indication being treated. Non-limiting examples of such medications include antibiotics, chemotherapeutic agents, aspirin, and sugammadex. Thus, in another embodiment, there is provided a syringe 10 in accordance with any of the above described embodiments, wherein the syringe comprises two or more panels, wherein each side of each panel is labeled with an independently selected indication and further comprises a scale, graduations, and indicia corresponding to the dose regimen appropriate to the indication. In one such embodiment, both sides of each panel may be labeled for the same indication. In another such embodiment, each side of each panel is labeled for a different independently selected indication.

By way of further illustration, the drug sugammadex is approved for two indications: for the reversal of deep neuromuscular blockade induced by rocuronium bromide and vecuronium bromide in adults and for the reversal of moderate neuromuscular blockade induced by rocuronium bromide and vecuronium bromide in adults. US FDA Prescribing Information, BRIDION® (sugammadex) Injection, for intravenous use Initial U.S. Approval: 2015. The approved label instructs the relevant heath care worker (e.g., anesthesiologists) to select one of two dosing scales based on the level of a patient's neuromuscular block (2 mg/mL vs 4 mg/mL) at the time of administration. Accordingly, in a non-limiting illustration of this embodiment, there is provided a syringe 10 in accordance with any of the above described embodiments, wherein the syringe comprises first and second panels 14, 114 which include two different (separate) dosing scales labeled as “deep neuromuscular block” and “moderate neuromuscular block”, respectively. The first and second scales 16, 116, are each independently marked with the appropriate graduations 62, 162, and with the appropriate indicia 64, 164, to represent the proper dose as a function of patient weight and the indication for which the drug is to be administered. In use, the attending health care worker selects the panel of the syringe based on the patient's indication (deep (4 mg/mL) or moderate (2 mg/mL) neuromuscular block) at the end of a surgery. The user fills the syringe based on the patient's weight, using the first or second graduations 62, 162 that display the appropriate dose for the chosen indication.

### Example 2: Differentiating Dose Markings for Adult and Pediatric Use

In some cases, a particular medication formulated for injection will be administered for two or more patient populations, each at different doses depending upon the patient type to which the drug is administered. For example, a particular drug may be administered to adult and pediatric patients, each at different dose rates. Thus, in another embodiment, there is provided a syringe 10 in accordance with any of the above described embodiments, wherein the syringe comprises a first panel 14 labeled for adult use, which first panel 14 is marked with a first scale 16 having the appropriate graduations 62 and the appropriate indicia 64 for adult dosing of the drug; and further comprising a second panel 114 labeled for pediatric use, which second panel 114 is marked with a second scale 116 having the appropriate graduations 162 and the appropriate indicia 164 for pediatric administration of the drug. In each panel the appropriate units of measure based on patient weight (e.g., km, mL) or patient surface area (e.g., m²) are indicated in the respective scale. The two (or more) panels may optionally be differentiated in any suitable way, including but not limited to labeling, color, branding, and the like.

By way of further illustration, the drug PEGINTRON® (peginterferon alfa-2b) injection is an antiviral medication administered by injection and indicated for the treatment of Chronic Hepatitis C (CHC) in patients with compensated liver disease. US FDA Prescribing Information, PEGINTRON® (peginterferon alfa-2b) injection U.S. Approval: 2001. According to its approved US label, peginterferon alfa-2b injection is approved for use in adult and pediatric patients. Accordingly, in a non-limiting illustration of this embodiment, there is provided a syringe 10 in accordance with any of the above described embodiments, wherein the syringe 10 comprises a first panel 14 labeled for adult use, which first panel 14 is marked with a first scale 16 having the appropriate graduations 62 and the appropriate indicia 64 for adult administration of (peginterferon alfa-2b) injection once weekly and wherein the adult dose rate is displayed in terms of patient weight (e.g., 1.5 micrograms drug per kg patient weight); and further comprising a second panel 114 labeled for pediatric use, which second panel 114 is marked with a second scale 116 having the appropriate graduations 162 and the appropriate indicia 164 for pediatric administration of (peginterferon alfa-2b) injection and wherein the pediatric dose rate is displayed in terms of patient body surface area (e.g., 60 micrograms of drug per m² of patient). In each panel the appropriate units of measure based on patient weight (e.g., km, mL) or patient surface area (e.g., m²) are indicated in the respective scale. The two (or more) panels may be arranged in any configuration described herein and may further and optionally be marked for easy identification and differentiation in any suitable way, including but not limited to labeling and color.

In a related embodiment, and by way of further illustration, certain pediatric medications are dosed according to the body surface area (BSA) of the pediatric patient. In such embodiments, the scale of the syringe 10 according to the invention and comprising at least one panel wherein at least one side of the at least one panel comprises a scale wherein the gradations and indicia are spaced in accordance with the approved dose rate as a function of surface area of the pediatric patient group to whom it is to be administered. In such embodiments, the BSA may be calculated by using the patient's weight and height using one of the following formulas (or other similar formulas):

$$( 1 ) BSA = \sqrt{\frac{Body Weight ( kg ) * Height ( cm )}{3600}} ( Mosteller Formula )$$

$$( 2 ) BSA = ( {W}^{0.425} \times {H}^{0.725} ) \times 0.007184 ( Du Bois , Du Bois Formula )$$

In such embodiments, doses are displayed on the panel in terms of surface area (e.g., square meters, square feet, etc.) on the syringe 10. By way of further illustration, the drug

### Example 3: Differentiating Dose Markings for Patient Age or Weight

The Center for Drug Evaluation and Research generally divides the pediatric population into the following groups: Neonates: birth up to 1 month; Infants: 1 month up to 2 years; Children: 2 up to 12 years; and Adolescents: 12 years up to 16 years. In such populations, dose is not necessarily linearly correlated with patient age or body weight; thus, medications indicated for these patient populations are administered at different dose rates depending on the patient group. Non-limiting examples of medications having different adult and pediatric dose rates are shown in the table below.

| | Therapeutic | | Pediatric |
|---|---|---|---|
| Drug | Indication | Adult Dose | Dose |
| Chloramphenicol | Bacterial | 50 mg | 50 mg |
| | infection | kg⁻¹ day⁻¹ | kg⁻¹ day⁻¹ |
| | | | neonates: 25 |
| | | | mg kg⁻¹ day⁻¹ |
| Carbamazepine | Epilepsy | 5-8 mg | >12 years: |
| | | kg⁻¹ every | 5-8 mg kg⁻¹ |
| | | 12 h | every 12 h |
| | | | Children: |
| | | | 3-10 mg |
| | | | kg⁻¹ every 8 h |
| | | | Infants: 3-10 |
| | | | mg kg⁻¹ every |
| | | | 8 h |
| Phenytoin | Epilepsy | 2 mg | Children: 2.3- |
| | | kg⁻¹ every | 2.6 mg⁻¹ kg |
| | | 12 h | every 8 h |
| | | | Infants: 2.3 |
| | | | mg kg⁻¹ |
| | | | every 8 h |
| | | | Neonates: |
| | | | 2.5-4.0 mg |
| | | | kg⁻¹ every 12 h |
| Propofol | Anaesthesia | <55 years: | 2 months-16 |
| | | 6-12 mg | years: 7.5-18 |
| | | kg⁻¹ h⁻¹ | mg kg⁻¹ h⁻¹ |
| | | >55 years: | |
| | | 3-6 mg | |
| | | kg−1 h−1 | |
| Busulfan | Cancer | 0.8 mg | ≤12 kg: 1.1 |
| | | kg⁻¹ every | mg kg⁻¹ |
| | | 6 h | every 6 h |
| | | | >12 kg: 0.8 |
| | | | mg kg⁻¹ every |
| | | | 6 h |
| Tobramycin | Bacterial | 3 mg | Children: |
| | infection | kg⁻¹ day⁻¹ | 6-7.5 mg |
| | | | kg⁻¹ day⁻¹ |
| | | | <2 weeks: 4 |
| | | | mg kg⁻¹ day⁻¹ |
| | | | With cystic |
| | | | fibrosis: 10 |
| | | | mg kg⁻¹ day⁻¹ |

Thus, in another alternative of the above embodiments, there is provided a syringe 10 in accordance with any of the above described embodiments comprising two or more panels, wherein each panel (or each side of each panel) is marked with a scale having the appropriate graduations and the appropriate indicia for at least one or more of neonatal administration, infant administration, children administration, and adolescent administration of a drug, wherein, in each panel the appropriate units of measure based on patient weight (e.g., km, mL) or patient surface area (e.g., m²) or other chosen parameter are indicated in the respective scale. The two (or more) panels may be arranged in any configuration described herein and may further and optionally be marked for easy identification and differentiation in any suitable way, including but not limited to labeling and color. By way of further non-limiting illustration, if a particular medication has a different dose rate for children and for adolescents, the dose rate for children could appear on one side of a panel and the rate for adolescents could appear on the other.

### Example 4: Differentiating Dose Markings for Normal and Impaired Organ Function

For some drugs, lower dose rates are indicated for individuals with impaired organ function, such as, kidney or liver function. This generally occurs when a medication or its metabolites depend on the kidney or liver for excretion and may add additional burden to the organ. Non-limiting examples of medications for which different dosing is required depending on the state of renal impairment include ACE inhibitors (e.g., Benazepril (Lotensin), Captopril (Capoten), Enalapril (Vasotec), Fosinopril (Monopril), Lisinopril (Zestril), Quinapril (Accupril), Ramipril (Altace)); Beta blockers (e.g., Acebutolol (Sectral), Atenolol (Tenormin), Bisoprolol (Zeberta), Nadolol (Corgard)); and Diuretics (e.g., Amiloride (Midamor), Burnetanide (Bumex), Furosemide (Lasix), Metolazone Zaroxolyn), Spironolactone (Aldactone), Thiasidesil, Torsemide (Demadex), and Triaamterene (Dyresium)). Munar & Sing, American Family Physician, vo. 75, no. 10, 2007. Kidney function is generally assessed based on creatinine clearance measurements in a blood test. Typically, dose rates are differentiated for individuals with creatinine clearance of <50 mL/min, but other thresholds may apply. Thus, in another embodiment, there is provided a syringe 10 in accordance with any of the above described embodiments comprising at least one panel 14 having a scale 16 and graduations 62 and indicia 64 each suitable to indicate the dose rate for the selected drug in non-organ impaired patients, and at least one additional panel having a scale, graduations and indicia indicating the dose rate for selected organ impaired patients. The scales for “normal” and impaired dose may each be displayed on different panels of the syringe 10, or on opposing sides of the same panels, respectively. As will be readily appreciated by those of ordinary skill in the art, the respective scales will be displayed in units appropriate to the medication (e.g., kg, mL, m²).

### Example 5: Differentiating Dose Markings for Multiple Medication Concentrations

Some medications come in multiple strengths and concentrations for different indications. Thus, in another embodiment, there is provided a syringe 10 in accordance with any of the above described embodiments comprising one or more panels, wherein each panel is marked with a scale having the appropriate graduations and indicia for dosing the selected drug in accordance with the strength or concentration. The syringe according to the invention may advantageously be used to reduce or prevent dosing error, with the dose rates corresponding to each medication concentration being displayed on each panel of the syringe 10. By way of further non-limiting example, the drug PEGINTRON® (peginterferon alfa-2b) for injection discussed above is available in multiple concentrations, e.g.: 50 mcg per 0.5 mL, 80 mcg per 0.5 mL, 120 mcg per 0.5 mL, 150 mcg per 0.5 mL in single-use vials (with 1.25 mL diluent). In one such embodiment, there is provided a syringe 10 in accordance with any of the above described embodiments comprising one or more panels comprising two or more scales for each of two or more such concentrations displayed thereon. In variations of such embodiments, the different scales are displayed on the same or different panel(s) as described herein.

### Example 6: Differentiating Dose Markings for Initial and Increased Dose Rate Due to Drug Resistance or Tolerance

Some medications may have two or more dose rates: one for new-users, and a higher one for users who have developed resistance or tolerance to the medication over time. Thus, in another embodiment, there is provided a syringe 10 in accordance with any of the above described embodiments comprising one or more panels wherein the dosing rate for the initial use is shown on at least one panel having a scale, gradations, and indicia indicative of the appropriate dose for initial use, and further comprising at least one additional scale, wherein the at least one additional scale is shown in the opposite side of the first panel or on the side of one or more additional panels each having a scale, gradations, and indicia indicative of the appropriate dose for subsequent use. By way of further illustration, the drug RENFLEXIS™ (infliximab-abda) for injection is a TNF alpha inhibitor indicated for the treatment of Chrone's disease, pediatric chrone's disease, ulcerative cholitis, rheumatoid arthritis, and other related indications. Renflexis™ is initially dosed at either 3 mg/kg or 5 mg/kg, depending on the indication. However, higher subsequent doses are indicated in some patients over time. In such patients, the dose can be increased to 10 mg/kg. In such embodiments, there is provided a syringe 10 in accordance with any of the above described embodiments comprising two or more scales, wherein a first such scale comprises the gradations and indicia indicative of the dose rate for patients who have not developed drug resistance or tolerance, and wherein the second scale comprises the gradations and indicia indicative of the dose rate for patients for whom higher approved doses are indicated.

### Example 7: Differentiating Dose Markings for Actual and Lean Body Weight

In some drugs, doses are prescribed or indicated as a function of a patient's body weight. Thus, in another embodiment, there is provided a syringe 10 in accordance with any of the above described embodiments comprising at least one panel 14, wherein said at least one panel comprises a scale having the appropriate gradations and indicia dosed according to patient body weight. Such body weight may be expressed as actual body weight or lean body weight in accordance with the prescribing information. By way of further illustration, the drug sugammadex, discussed above, is administered as a one-time injection at a dose of either 2 mg/kg or 4 mg/kg of patient weight, and the medication is dispensed in 100 mg/mL concentrations. In such embodiments, the syringe 10 comprising at least one panel 14 is configured with the appropriate scale, gradations and indicia to display medication volume in terms of patient weight, such that the following dosing scales (e.g., in kg) are displayed on the face of either of two panels of the syringe 10:

| | First Panel | Second Panel |
|---|---|---|
| | 14 (First | 114 (Second |
| | scale 16) | scale 116) |
| Volume | (2 mg/kg) | (4 mg/kg) |
| 0.8 mL | 40 kg | |
| 1.0 mL | 50 kg | |
| 1.2 mL | 60 kg | |
| 1.4 mL | 70 kg | |
| 1.6 mL | 80 kg | 40 kg |
| 1.8 mL | 90 kg | |
| 2.0 mL | 100 kg | 50 kg |
| 2.2 mL | 110 kg | |
| 2.4 mL | 120 kg | 60 kg |
| 2.6 mL | 130 kg | |
| 2.8 mL | | 70 kg |
| 3.0 mL | | |
| 3.2 mL | | 80 kg |
| Etc. | | |

### Example 8: Differentiating Dose Markings in Terms of Patient Ideal Weight

In a related embodiment, “ideal weight,” rather than the actual weight of the patient, is used to calculate an appropriate dose in overweight and obese patients, particularly in instances where the administered drug is not fat soluble. In such cases, dosing by body weight alone would cause an overdose of overweight and obese patients, because the drug would distribute in the non-fatty regions of the body at supra-therapeutic concentrations. Thus, in another embodiment, the patient “ideal weight” may be displayed on the syringe 10 in addition to, or instead of, real body weight. Non-limiting examples of drugs dosed according to patient ideal weight include Acyclovir, Amikacin, Atracurium, Colistin, Dalteparin, Daptomycin, Digoxin, Dobutamine, Dopamine, Enoxaparin, Epinephrine, Fondaparinux, Gentamicin, Heparin (unfractionated), Immunoglobulin (IVIG), Levothyroxine, Linezolid, Lorazepam, Methylprednisolone, Midazolam, Norepinephrine, Oseltamivir, Phenytoin, Propofol, Rasburicase, Remifentanil, Rocuronium, Succinylcholine, Tigecycline, Tinzaparin, Tobramycin, Vancomycin, Vecuronium, Voriconazole and Warfarin.

### Example 9: Differentiating Dose Markings to Display Minimum and Maximum Dose

Some medications have minimal effective doses, and all medications have maximum doses that can be administered without a toxic effect. Thus, in another embodiment, there is provided a syringe 10 in accordance with any of the above described embodiments comprising at least one panel 14, wherein said at least one panel comprises a scale having the appropriate gradations and indicia that displays the minimum dose together with the variable-dependent dosing scale. In another such embodiment, the scale displays the maximum dose together with the variable-dependent dosing scale, and in another such embodiment, the scale displays the minimum and maximum dose together with the variable-dependent dosing scale. Such embodiments may advantageously make minimum and/or maximum dosing limits more obvious to the person administering the medication.

By way of further illustration, the drug haloperidol decanoate is approved for injection for the long term treatment of certain mental and mood disorders such as schizophrenia. According to approved labels, the maximum volume of haloperidol decanoate per injection should not exceed 3 mL, and the maximum dose per injection should not exceed 100 mg. Thus, in such embodiments, either the maximum volume or the maximum dose (or both) may be displayed on at least one side of a panel 14 of the syringe 10 according to the invention.

### Example 10: Differentiating Dose Markings to Display Dosing Scale for Drugs Exhibiting Non-Linear Pharmacokinetics

Typically, blood concentrations of medications are assumed to follow a nearly-linear dependence on dose. However, a concentration/dose relationship can become non-linear when the medication's bioavailability, fraction of unbound drug in the blood, and clearance rate change based on the medication's blood concentration. When a drug exhibits non-linear pharmacokinetics, these parameters are usually dependent on the reaction rates of steps in the drug metabolism process. If a reaction has a maximum rate, or if a critical enzyme becomes saturated, drug may accumulate in the blood stream at a higher rate at higher drug doses. In such cases, administering increasing volumes of medication may require a relatively higher or lower dose rate than the original rate to achieve or maintain a drug's effect or desired blood concentration. Drugs are typically not dosed this way because appropriate doses are difficult to predict and calculate. A syringe 10 according to this embodiment may advantageously reduce the error rate or increase the convenience of such dosing. Thus, in another embodiment, there is provided a syringe 10 according to the invention comprising at least one panel 14, wherein said at least one panel comprises a scale having the appropriate graduations and indicia showing the calculated increased (or decreased) dose in terms of weight or another appropriate unit.

Erythropoietin is a drug or class of drugs used by injection for the treatment of various indications including anemia. Erythropoietin has been shown to be eliminated from the body through a series of non-linear pathways. Veng-Pedersen et al., J Pharm Sci. 1995 June; 84(6):760-7. In this study, clearance was shown to decrease significantly (and therefore accumulation and medication exposure increased significantly and non-linearly) at doses of 100 and 500 U/kg compared to 10 U/kg. This non-linearity occurs once blood concentration exceeds the concentration required to saturate the drug's metabolic pathways. Thus, in another embodiment, there is provided a syringe 10 according to the invention comprising at least one panel, said panel having a scale with gradations and indicia suitable to display decreasing marginal volume per unit weight at dosages above the saturation threshold to account for decreasing amount of medication needed to achieve an additional medicinal effect.

### Example 11: Differentiating Dose Markings to Display Pediatric Dose Based on Allometric Scaling

While most pediatric doses for drugs are calculated by linearly scaling adult doses down to pediatric ranges (the scaling ratio is usually calculated either by body weight or body surface area, e.g., as described above), this method may lack desired accuracy, as differences in metabolic capacity do not always scale linearly with body size or weight, and may either under- or over-dose pediatric patients. Several studies have shown this method to more accurately predictor drug clearance and drug metabolism than conventional models in a variety of medications, including morphine, fentanyl, and remifentanil. Cella M, et al., Br J Clin Pharmacol, 2010. Mahmood, I., Ther Drug Monit. 2007; 29:271-8. Mahmood, I., Br J Clin Pharmacol. 2006; 61:545-57. Anderson B J, et al., Annu. Rev. Pharmacol. Toxicol. 2008; 48:303-32. The allometric exponent can either be assumed constant, or can be calculated on a per-drug basis. Allometric scaling is an alternative to linear dosing which has been shown to be the most accurate dosing method in many examples. Allometric scaling follows the following relationship:

$${P}_{c h i l d} = {{P}_{adults} ( \frac{W T}{70} )}^{x}$$

where P is the parameter of interest (e.g., drug clearance or dose, WT is the bodyweight of the individual child and x is the allometric exponent. Allometric scaling has not gained widespread acceptance as standard practice due to continued debate as to modeling best practices. Further, even where allometric scaling is desired for use, difficulties in performing the appropriate dose for a given drug can impede its use at the point of use. Where allometric scaling is desired, a syringe according to the invention can advantageously reduce the impediments to use related to difficulties of calculation by including the allometric dose scaling for the particular drug on the panel of the syring. Thus, in another embodiment, there is provided a syringe 10 according to the invention comprising at least one panel, wherein said at least one panel comprises a scale having the gradations and indicia displayed for allometric dosing. Such doses may be displayed in the appropriate units (e.g., kg, m², etc.).

What is claimed is:

1. A syringe for injecting medication into a patient, the syringe comprising:
a tubular barrel having opposing inner and outer surfaces, an open proximal end, and a distal end having a wall extending thereacross, an opening being defined in said wall with a needle mount extending distally from said wall about said opening;
a first panel extending radially outwardly from said outer surface of said barrel, said first panel having opposing first and second faces; and,
a first scale including a series of spaced-apart first graduations disposed on said first face of said first panel.

2. A syringe as in claim 1, wherein each of said first graduations disposed on said first face of said first panel extends uninterruptedly at least partially about a circumference of said barrel.

3. The syringe of claim 1, further comprising a second panel extending radially outwardly from said outer surface of said barrel, said second panel having opposing first and second secondary faces.

4. The syringe of claim 1, further comprising a second scale including a series of spaced-apart secondary graduations disposed on said first secondary face of said second panel.

5. The syringe as in claim 4, wherein said secondary graduations are spaced-apart at different intervals from said first graduations.

6. The syringe of claim 4, wherein said first face and said secondary first face both face in generally the same direction.

7. The syringe of claim 3, wherein said second panel extends from said outer surface of said barrel in a generally diametrically opposing direction from said first panel.

8. The syringe of claim 1, further comprising a flange disposed on said outer surface of said barrel about said proximal end.

9. The syringe as in any of claim 1, wherein said first panel includes a plurality of edges extending between said first and second faces, a first of said edges facing proximally, a second of said edges facing distally, and a third of said edges extending between said first and second edges, said first edge being spaced distally from said flange to define a space therebetween.

10. The syringe of claim 8, wherein said flange having first and second flange portions extending radially outwardly from said outer surface of said barrel in opposing directions, said third edge being located radially outwardly a first distance from said outer surface of said barrel, said first flange portion extending radially outwardly a second distance from said outer surface of said barrel, said first distance being 80%-150% of said second distance.

11. The syringe of claim 1, wherein said first panel is radially aligned with said first flange portion on said outer surface of said barrel.

12. The syringe of claim 1, wherein said first scale being presented based on at least one physiological parameter.

13. The syringe as in claim 12, wherein said physiological parameter is selected as one or more from a group consisting of: age of a patient, gender of a patient, actual weight of a patient, ideal weight of a patient, body surface area of a patient, severity of a patient's condition, level of impairment of a patient's one or more organs, concentration of a medication, patient's resistance to a medication, pharmacokinetics of a medication, and allometrically scaled dosing of a medication.

14. The syringe of claim 1, wherein said first scale increases in magnitude in a proximal direction.

15. The syringe of claim 1, wherein said barrel is formed of thermoplastic.

16. The syringe of claim 1, wherein the syringe is a plastic disposable syringe.

17. The syringe of claim 1, further comprising a plunger formed to be inserted into said barrel through said proximal end.

18. The syringe of claim 1, wherein said first panel is unitarily formed with said barrel.

19. The syringe of claim 1, wherein said first panel has a planar surface.

20. The syringe of claim 3, wherein said second panel has a planar surface.
